(19) United States
(12) Patent Application Publication
(10) Pub. No.: US 2006/0100517 A1
(43) Pub. Date: May 11, 2006

(54) Integrated pressure and volume measurement methods and apparatus

(75) Inventor: Robert Allan Phillips, New South Wales (AU)

Correspondence Address: MERCHANT & GOULD PC, P.O. BOX 2903, MINNEAPOLIS, MN, 55402-0903, US

(73) Assignee: USCOM Pty Ltd., New South Wales 2450 (AU)

(21) Appl. No.: 10/544,332

(22) PCT Filed: Jun. 26, 2003
(86) PCT No.: PCT/AU03/00808

§ 371 Date: Dec. 29, 2004

(30) Foreign Application Priority Data
Jul. 3, 2002 (AU) PS 3353

(51) Int. Cl. A61B 8/02

(52) U.S. Cl. 600/450; 600/456

(57) ABSTRACT

An apparatus for measuring pressure volume loop parameters of a patient (10), the apparatus comprising: an intra cardiac blood flow measurement device (11) for producing at least a first output indicative of the blood flow of a patient (10); a pressure sensor means (14) producing at least a second output indicative of the blood flow pressure of a patient (10); and a processing element (12) for combining at least the first and second outputs to produce at least one third output indicative of the operation of the pressure volume loop of the patient (10).

### FIELD OF THE INVENTION

The present invention relates to the measurement of bodily functions arid, in particular, to the utilisation of integrated blood pressure and blood volume measurements in a non-invasive manner.

### BACKGROUND OF THE INVENTION

The relationship between blood pressure and blood volume flows within the body is complex and is an extremely important interrelationship.

In particular, large scale analysis of pressure volume loop characteristics has been undertaken by cardiologists in determining cardiac output. The area of the pressure volume loop represents the work done during a stroke of the heart during each heart beat. Detailed analysis of the interaction between volume, flow and pressures in the body is extremely important.

Currently measurements of flow, volume and pressure interactions involve invasive patient measurement. It would be desirable to provide for a simplified form of pressure, volume and flow measurements of blood flows within the body.

### SUMMARY OF THE INVENTION

It is an object of the present invention to provide an improved system for blood pressure volume loop analysis.

In accordance with a first aspect of the present invention there is provided an apparatus for measuring pressure volume loop parameters of a patient, the apparatus comprising: an intra cardiac blood flow measurement device for producing at least a first output indicative of the blood flow of a patient; a pressure sensor means producing at least a second output indicative of the blood flow pressure of a patient; and a processing element for combining at least the first and second outputs to produce at least one third output indicative of the operation of the pressure volume loop of the patient.

Preferably the intra cardiac blood flow measurement device comprises a CW Doppler flow measurement transducer device for emitting and receiving a CW Doppler signal and a signal processing means for extracting blood flow information from the received signal. Further, the pressure sensor means preferably produces continuous repetitive pressure measurements.

In accordance with a further aspect of the present invention, there is provided a method of measuring pressure volume loop parameters of a patient comprising the steps of: obtaining at least one measure of blood flow within the heart of a patient utilising CW Doppler methods; obtaining at least one measure of blood flow pressure within the vessels of the patient; and combining the measures of step (a) and step (b) to produce the pressure volume loop parameters.

In accordance with a further aspect of the present invention, there is provided a method of creating a pressure volume loop parameter index comprising the steps of: (a) obtaining measures of blood flow within the heart of a patient utilising CW Doppler methods; (b) obtaining at least one measure of blood flow pressure within the vessels of the patient; (c) combining the measures of step (a) and step (b) to produce the pressure volume loop parameters; (d) repeating the step (c) for a plurality of patients to obtain a set of calculations; (e) processing the set of calculations to obtain a statistically averaged index across a population.

In accordance with a further aspect of the present invention there is provided a method of measuring pressure volume loop parameters of a patient comprising the steps of: utilising CW Doppler methods to obtain at least one measure of blood flow within vessels in a first predetermined volume of the body of a patient; obtaining at least one measure of blood flow pressure within the vessels of said first predetermined volume of the body of the patient; and combining the measures to produce pressure volume loop parameters for blood flow within said vessels.

### DESCRIPTION OF THE DRAWINGS

The preferred embodiments of the present invention will now be described with reference to the accompanying drawings in which:

FIG. 1 illustrates the utilisation of an apparatus constructed in accordance with the preferred embodiment;

FIG. 2 illustrates in more detail the transducer used in the preferred embodiments;

FIG. 3 illustrates a velocity time output from a CW Doppler signal;

FIG. 4 illustrates various components of the signal of FIG. 3;

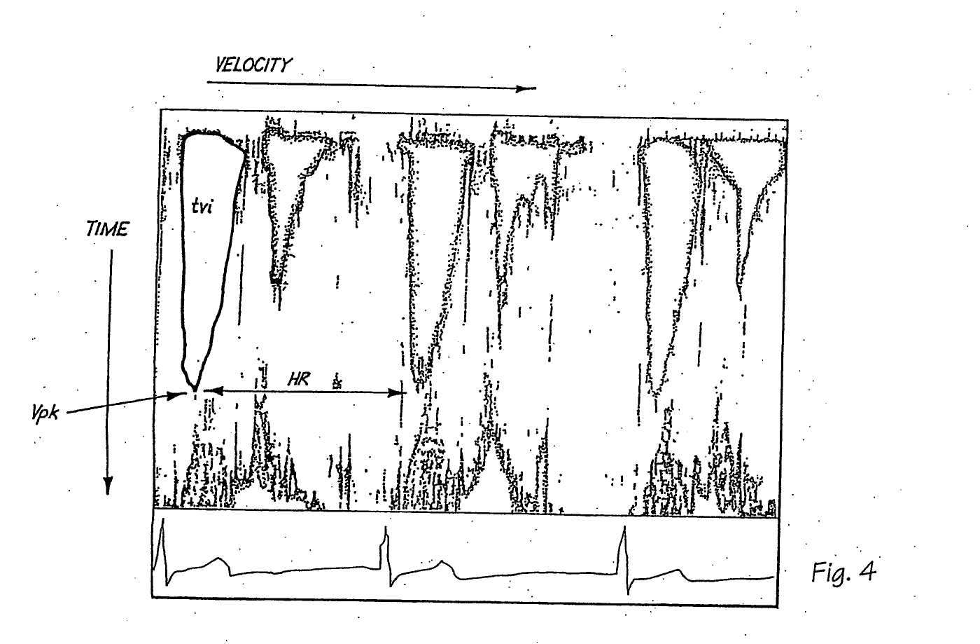

FIG. 5 illustrates a functional block diagram of a simplified form of processing apparatus of the preferred embodiments; and

FIG. 6 illustrates a flow chart of the steps involved in calculation of the pressure volume loop index.

### DETAILED DESCRIPTION OF THE PREFERRED AND OTHER EMBODIMENTS

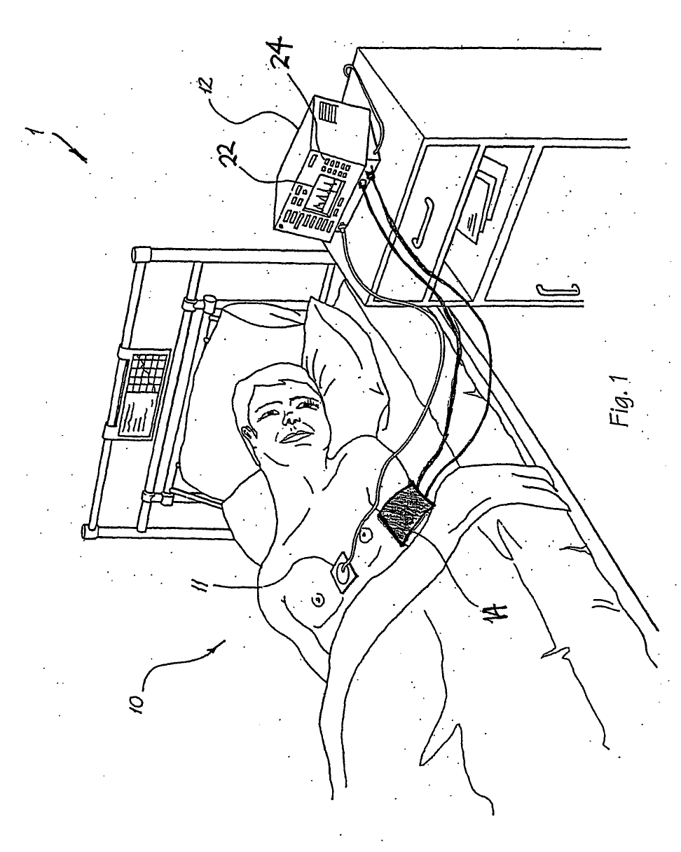

In the preferred embodiment, there is provided an integrated system for pressure and volume measurement of blood flows within the body. Turning to FIG. 1, there is illustrated the operational arrangement of the preferred embodiment 1, wherein a patient 10 has two non-invasive sensors including cardiac flow monitor 11 and pressure sensor device 14. Each of the two sensors 11, 14 are interconnected to a processing and display unit 12 located adjacent the patient 10. The processor unit 12 includes internal computer processing means, a display 22, and a series of control buttons e.g. 24 for controlling the functionality of the device. The unit 12 can in turn be interconnected in a network in the normal manner via an Ethernet connection or the like.

The pressure sensor 14 provides for blood pressure measurements for forwarding to the arrangement 12. The element 14 preferably allows for automatic blood pressure monitoring and can, for example, comprise suitably adapted version of the HEM757 automatic blood pressure monitor available from Quick Medical. The arrangement preferably provides for continuous blood pressure measurement of the patient 10.

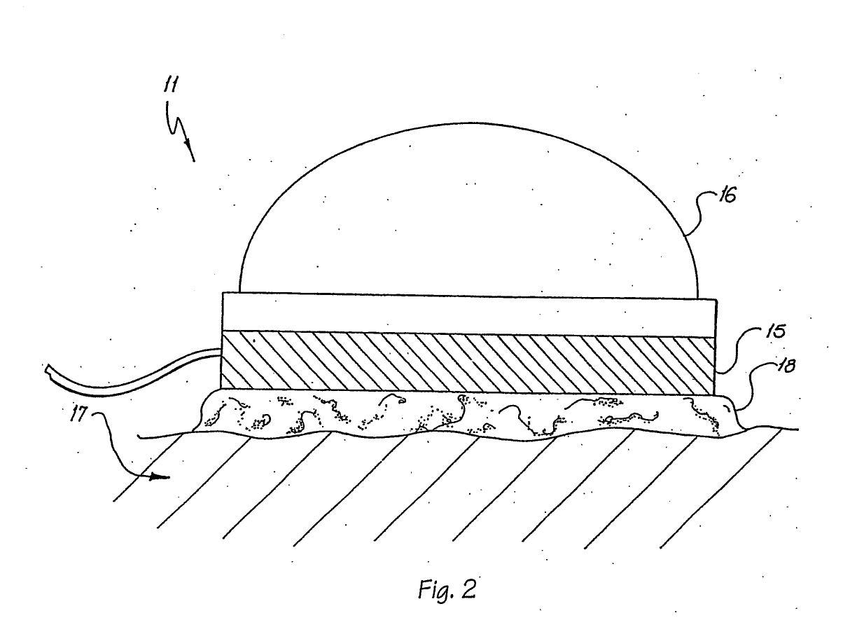

FIG. 2 shows an example of the first actuator 11 for attachment to the skin surface. Ideally CW Doppler is utilised to monitor blood flow. CW Doppler is a non-invasive technique in which ultrasonic signals from transducer elements are directed into a blood carrying vessel of a patient. Doppler shifts in the reflected signal provide an indication of the rate of blood flow. In FIG. 2, a transducer element 11 includes an ultrasonic transducer 15 attached to a positioning device 16 which can be used to initially set the position of the transducer. Between the transducer 15 and a patient's skin 17 is placed a gel coupling layer 18 for coupling the ultrasonic transducer vibrations to the skin 17. The principles of CW Doppler flow measurement are known. Patent Cooperation Treaty (PCT) publication number WO 99/66835 assigned to the present assignee, the contents of which are incorporated herein by cross-reference, describes in more detail an ultrasonic transducer device suitable for measuring blood flow using the CW Doppler method.

In the embodiment shown in FIG. 1, the transducer elements are placed on the patient to obtain intra-cardiac or aortic signals, for example through a suprastemal notch.

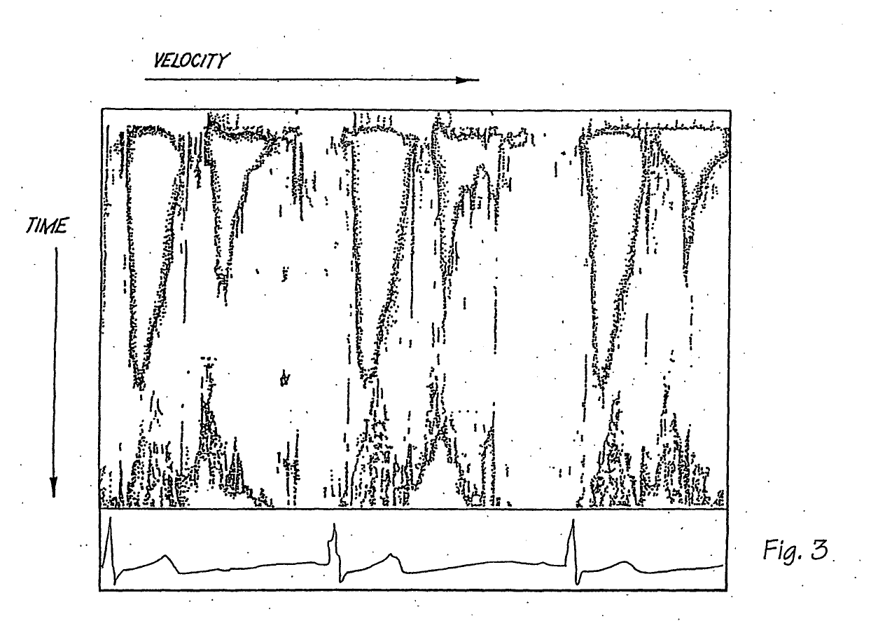

The CW method detects the velocity of individual blood cells by measuring the frequency change of a reflected ultrasound beam and displaying this as a velocity time flow profile, an example of which is shown in FIG. 3. The transducer output forms an input to the processor unit. From the velocity time flow profile, the processor calculates the velocity time integral (vti) and other relevant information such as heart rate (HR).

The processor unit receives the outputs of the blood flow monitor and the pressure measurement device. The heart rate may also be input into the processor from a separate heart rate monitor. Alternatively, the heart rate may be calculated from the blood flow profile, e.g. by counting the number of beat peaks over a period of time.

The processor mathematically combines its inputs to derive new parameters pertaining to the pressure volume loop. The processor can combine the results in many different ways so as to produce informative measures. For example, by division of the pressure values by the volume values. The results can be displayed on screen 22.

This combination of pressure and volume inputs further allows for derivation of indices reflecting contractile cardiac state and cardiac reserve such as peak and mean ventricular power and rate pressure product. The results of these calculations can be displayed real time. A variety of vascular indices can also be determined and displayed such as systemic arterial compliance, systemic vascular resistance which can be combined with central volume measurements to create cardiovascular indices such as stroke volume to aortic pulse pressure ratio. Sphygmomanometric measures can also be used for more robust but less continuous inputs of pressure such as systolic, diastolic and mean blood pressure.

Analysis based on these new heart rate/pressure/volume parameters provide for an improved understanding of physiology and pathophysiology associated with cardiovascular function, exercise and pulmonary function. They can also facilitate new methods of categorising conditions and new methods of diagnosing conditions within a patient. Furthermore, these new parameters can also provide greater monitoring of conditions within a patient, e.g. during recovery, as the parameters can provide complete indications of pressure-volume loop analysis which relates directly to the ongoing health of the tissue.

The above benefits may be realised by the creation of pressure volume loop indices. For example, a parameter produced by the combination of blood flow and pressure readings may be measured by a plurality of known healthy patients to determine an index for the parameter, which index denotes good is health. The pressure volume loop index is established by measuring the parameter in a plurality of healthy patients and then statistically averaging the results to determine a value or range of values of the relevant pressure volume loop parameter.

Similarly, the same parameter may be measured in a plurality of patients having a known condition to obtain a pressure volume loop index relating to that parameter, which index describes the presence of that condition.

Diagnosis of a condition in a patient can then be performed by calculating the parameter for the patient and comparing the result with the parameter indices to determine whether the condition exists in the patient.

Statistical averaging of the parameter values of individual patients can further take into account such variables as age, sex, height, weight, ethnicity etc. The indices thereby produced may be scaled according to these factors, e.g. the upper and lower limits of a pressure volume loop parameter denoting good health may vary with age or there may be a different range of values depending on sex.

As the processor unit is able to perform a real time calculation of a pressure volume loop parameter, the preferred embodiment of the present invention allows for continuous monitoring and healthcare of a patient based on the pressure volume loop reading. For example, a patient in recovery may have the pressure monitored in a relevant region of the body. If the pressure volume reaches a healthy range of the pressure volume loop of the index, then good health is indicated and treatment may be ceased or reduced. Conversely, the pressure volume loop reading of a patient may change to an unhealthy level. An alarm state may be triggered when the pressure volume loop reading crosses a threshold level so that corrective action, e.g. to the course of treatment, may be taken.

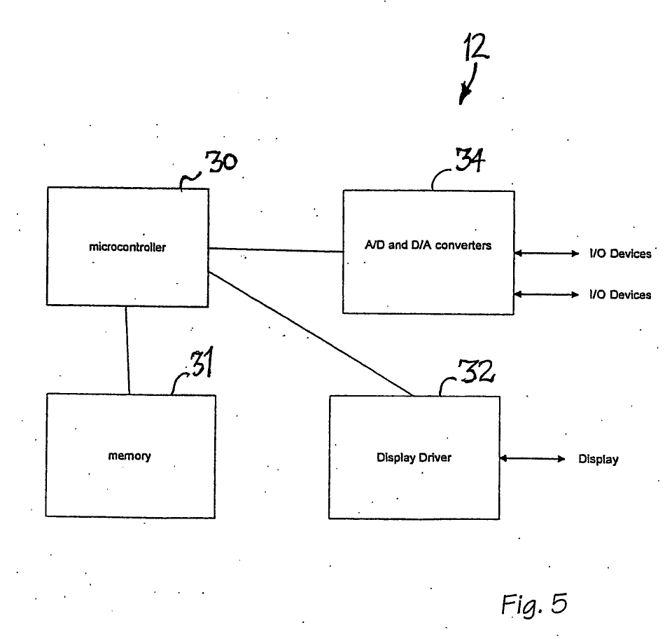

Turning to FIG. 5, there is illustrated one form of a standard functional block arrangement for a design of the device 12. The device 12 can be based around a microcontroller 30 which is driven by a software program stored either in onboard memory or in an external memory 31. The microcontroller 30 interacts with a display driver 32 for driving the display and a series of AID and D/A converters 34 for driving external devices. The 10 devices can include the transducer element 11 and pressure manipulation element and other sensors 14.

The microcontroller 30 operates primarily in accordance with software programming and the arrangement of FIG. 5 will be readily understood by those skilled in the art of design of digital hardware systems. Of course, depending on requirements, other hardware designs are possible. For example, the hardware could be designed around the programming of a standard Wintel type PC arrangement having separate cards for each of the pressure and volume sensors. Alternatively, fully custom ASIC based designs could be implemented.

The microcontroller 30 utilises a pre-processing step on the CW transducer signal input 11. The transducer output is detected and image processed utilising known techniques to determine the relevant heart flow parameters including the flow profile, velocity time integral, heart rate etc. The flow parameters may be calculated over successive frames, i.e. heartbeats either to obtain average readings or to determine variation in time base parameters. Simultaneously, the inputs from the pressure monitoring device are also provided and analysed to determine a current blood pressure reading. The respective results of this step are then combined by the microcontroller 30 to calculate the above parameters. The results may be then compared with pre-loaded results stored in memory 31 so as to determine, for example, whether an alarm condition exists. The results then can be displayed graphically on the display 22. Of course, other devices such as oximetry devices etc. can be also utilised for input to the processing unit 12. Other inputs can include other forms of heart rate monitors if the heart rate is not calculated directly from the blood flow profile.

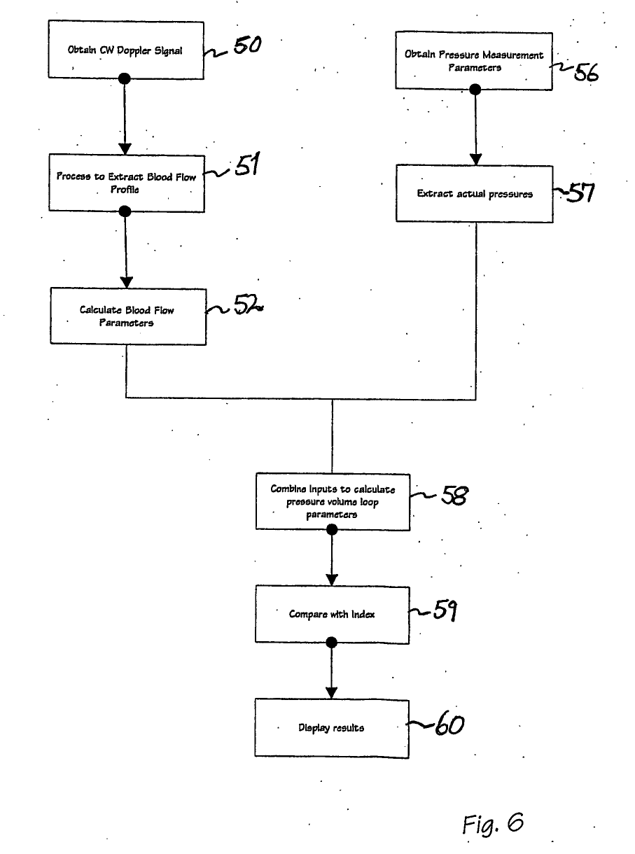

Turning now to FIG. 6, there is illustrated the steps involved in determining the various pressure volume loop measurements. It is noted previously, the CW doppler signal is obtained 50 and subsequently processed so as to extract the blood flow profile 51. From the blood flow profile, the blood flow parameters 52 are calculated. Simultaneously, the pressure measure parameters 56 are obtained and subsequently there is extracted the actual pressure measurement 57. The two inputs from stages 52 and 57 are combined 58 so as to determine pressure volume loop parameters which are then compared with an index 59 and the results output 60 to a display device.

In order to establish the index, the steps 50-60 are repeated on a plurality of patients and the total set of results statistically combined, e.g. averaged for patient variables such as age, sex, weight etc.

Further, the measurements can also be carried out whilst the patient is undergoing a number of different physical conditions, for example, sleeping, walking and running. The measurements may be combined in a similar manner to produce subindicies for each type of activity for each person type.

While the embodiments described relate to combined intra cardiac or aortic flow and pressure measurement, it is further possible to combine any vessel flow with pressure measurement to determine isolated pressure volume loop parameters in a relevant area of the body. Other examples include femoral flow, cubital or maxillary flow or cartoid flow. Such devices have multiple diagnostic applications in the safe and cost effective delivery of health care to humans and animals, particularly in the emergency room, operating theatre, paediatric surgery, sleep medicine and in the management of heart failure. Another area of utilization may be sports medicine

Additionally, the invention as described herein can be used to improve understanding of the normal physiology and pathophysiology associated with cardiovascular function, exercise and pulmonary function.

It will be understood that the invention disclosed and defined herein extends to all alternative combinations of two or more of the individual features mentioned or evident from the text or drawings. All of these different combinations constitute various alternative aspects of the invention.

The foregoing describes embodiments of the present invention and modifications, obvious to those skilled in the art can be made thereto, without departing from the scope of the present invention.

1. An apparatus for measuring pressure volume loop parameters of a patient, said apparatus comprising:
an intra cardiac blood flow measurement device for producing at least a first output indicative of the blood flow of a patient;
a pressure sensor means producing at least a second output indicative of the blood flow pressure of a patient; and
a processing element for combining at least said first and second outputs to produce at least one third output indicative of the operation of the pressure volume loop of the patient.

2. An apparatus as claimed in claim 1 wherein said intra cardiac blood flow measurement device comprises a CW Doppler measure measurement transducer device of emitting and receiving a CW Doppler signal and a signal processing means for extracting blood flow information from said received signal.

3. An apparatus as claimed in claim 1 wherein said pressure sensor means produces continuous repetitive pressure measurements.

4. A method of measuring pressure volume loop parameters of a patient comprising the steps of:
obtaining at least one measure of blood flow within the heart of a patient utilising CW Doppler methods;
obtaining at least one measure of blood flow pressure within the vessels of the patient;
combining the measures of step (a) and step (b) to produce said pressure volume loop parameters,

5. A method of creating a pressure volume loop parameter index comprising the steps of:
(a) obtaining measures of blood flow within the heart of a patient utilising CW Doppler methods;
(b) obtaining at least one measure of blood flow pressure within the vessels of the patient;
(c) combining the measures of step (a) and step (b) to produce said pressure volume loop parameters;
(d) repeating the step (c) for a plurality of patients to obtain a set of calculations;
(e) processing said set of calculations to obtain a statistically averaged index across a population.

6. A method as claimed in claim 5 her comprising the step of utilizing said statistically averaged index to correlate subsequent measurement of pressure volume loop parameters to determine health characteristics of a patient.

7. A method as claimed in claim 6 wherein said method is separately carried out for patients of similar age, weight, sex or ethnic background.

8. A method as claimed in claim 6 wherein said method is separately carried out for patients whilst the patient undertakes different levels of physical activity.

9. A method of measuring pressure volume loop parameters of a patient comprising the steps of:
(a) utilising CW Doppler methods to obtain at least one measure of blood flow within vessels in a first predetermined volume of the body of a patient;
(b) obtaining at least one measure of blood flow pressure within the vessels of said first predetermined volume of the body of the patient;
(c) combining the measures of step (a) and step (b) to produce said pressure volume loop parameters for blood flow within said vessels.

10. (canceled)
